(19) United States
(12) Patent Application Publication
(10) Pub. No.: US 2019/0040293 A1
(43) Pub. Date: Feb. 7, 2019

(54) LATENT HEAT STORAGE MEDIUM

(71) Applicant: i-select SA, Neuchâtel (CH)

(72) Inventor: Olaf ANDERS, Witzenhausen (DE)

(73) Assignee: i-select SA, Neuchâtel (CH)

(21) Appl. No.: 16/048,533

(22) Filed: Jul. 30, 2018

(30) Foreign Application Priority Data
Aug. 3, 2017 (DE) 10 2017 117 599.7

(51) Int. Cl. C09K 5/06; C08F 20/56; C07C 51/50

(52) U.S. Cl. CPC C09K 5/063; C08F 2810/20; C07C 51/50; C08F 20/56

(57) ABSTRACT

The present invention makes available an additive for stabilization of sodium acetate trihydrate, which can be obtained by emulsion polymerization of at least one hydrophilic monomer selected from among acrylic acid and acrylic acid derivatives with an acid group and a redox initiator system. Furthermore, a method for producing the additive, a storage medium of a latent heat storage unit, containing sodium acetate trihydrate and the additive, a method for stabilizing a storage medium of a latent heat storage unit, as well as the use of the additive as a stabilizer for sodium acetate trihydrate in a latent heat storage unit are made available.

### BACKGROUND OF THE INVENTION

### Field of the Invention

The invention relates to an additive for stabilization of sodium acetate trihydrate, as well as to a method for production of this additive. The invention furthermore relates to a storage medium for a latent heat storage unit, comprising sodium acetate trihydrate and the additive, as well as to a method for stabilization of this storage medium.

### Background of the Invention

Latent heat storage units are known in the state of the art. These possess the ability to store thermal energy over an extended period of time and with a great number of repeat cycles. The basic principle of the latent heat storage unit is based on utilization of the enthalpy of thermodynamic state changes, such as the phase transition from liquid to solid and vice versa, for example. In latent heat storage units, sodium acetate trihydrate (CH₃COONa*3H₂O), a colorless salt hydrate that dissolves well in water at 20° C., is used.

Sodium acetate trihydrate is a solid substance at room temperature. It is liquefied by heating it above the melting temperature of 58° C. After complete liquefaction of the material, it can be cooled down even to temperatures far below the melting point, for example to room temperature or, under some circumstances, as far as down to −20° C., without crystallizing. The sodium acetate trihydrate is then present as a supercooled melt, i.e. in a thermodynamically meta-stable state, in which a phase transition should actually take place. This effect is utilized, for example, in commercially available hand warmers or pocket warmers. In these, a small metal plate is generally found, which, after a change in state, then starts crystallization of the material, and gives off the energy released by the crystallization in the form of heat.

### DESCRIPTION OF THE INVENTION

Sodium acetate trihydrate is too unstable in the supercooled melt, without additional additives. Experiments have shown that spontaneous release occurs, among other things, in amounts of more than 200 kg. The amounts required for possible applications, for example storage of heat for hot water and heating, therefore need an additive in order to increase stability.

What is problematic in the case of supercooled melts on the basis of sodium acetate trihydrate is the circumstance that after some time and some repetition cycles, separation of the water can occur. Furthermore, increasing “anhydrate” formation can come about, which is further reinforced by the increasing concentration gradient. It is furthermore problematic that spontaneous release can also proceed from this so-called anhydrate, in other words crystallization brought about in targeted manner at a specific point in time is made difficult to impossible.

It is therefore important to create an environment for the storage medium sodium acetate trihydrate that is used, which environment keeps disruptive factors as low as possible. Furthermore, the environment is supposed to ensure that spontaneous release does not occur, or at least occurs at a clearly lower probability.

The stability of the supercooled melt and the specific storage capacity of the sodium acetate trihydrate presume that neither de-mixing of salt and water nor anhydrate formation comes about.

It is therefore the task of the present invention to make available a composition with which the problems stated above do not occur or at least can be reduced very greatly, in order to increase the stability of the sodium acetate trihydrate.

This task is accomplished by an additive for stabilization of sodium acetate trihydrate. This task is furthermore accomplished by a method for producing the additive, a storage medium for a latent heat storage unit, comprising sodium acetate trihydrate and the additive, a method for stabilization of a storage medium for a latent heat storage unit, as well as the use of the additive as a stabilizer for sodium acetate trihydrate in a latent heat storage unit.

The additive for stabilization of sodium acetate trihydrate can be obtained by means of emulsion polymerization of at least one hydrophilic monomer selected from among acrylic acid and acrylic acid derivatives with an acid group and a redox initiator system.

In this regard, the redox starter can be used in an amount of 0.1% to 10% with reference to the total mass of polymer including water.

In this regard, the monomer can comprise an acrylic acid derivative of sulfonic acid or of phosphonic acid or a salt of sulfonic acid or phosphonic acid.

The additive can be a monomer selected from the group comprising 2-acrylamido-2-methyl propane sulfonic acid, 2-methacrylamido-2-methyl propane sulfonic acid, 2-acrylamido-2-methyl-1-propane phosphonic acid, 2-methacrylamido-2-methyl-1-propane phosphonic acid, 2-(methacryloyloxy)ethyl-phosphonic acid, a salt thereof, or a mixture thereof.

Preferably, the monomer can be 2-acrylamido-2-methyl propane sulfonic acid.

In this regard, the additive can be obtained using a cross-linking agent in an amount of up to 8 mol-%, with reference to the amount of monomer and cross-linking agent.

The cross-linking agent can be selected from the group comprising ethylene glycol dimethyl acrylate, N,N-methylene bisacrylamide, pentaerythritol tetra-acrylate, trimethylolpropane trimethacrylate, trimethylolpropane triacrylate, and mixtures thereof.

Preferably, the amount range of the cross-linking agent lies at 0.2 to 0.6 mol-%.

Preferably, the cross-linking agent can be ethylene glycol dimethacrylate.

Preferably, the cross-linking agent comprises a mixture of ethylene glycol dimethacrylate and N,N-methylene bisacrylamide.

In this regard, the mixture of ethylene glycol dimethacrylate and N,N-methylene bisacrylamide can be used in any mixture ratio. Preferably, the mixture ratio lies at 1:3 to 3:1, particularly preferably at 2:3 ethylene glycol dimethacrylate to N,N-methylene bisacrylamide.

The redox initiator system can comprise a persulfate, a hydroperoxide or an Na or K hypochloride as an oxidation agent, and ascorbic acid, formaldehyde sulfoxylate, tetramethylene diamine, sodium hydroxymethylsulfinate, potassium pyrosulfite, sodium hydrogen sulfite, sodium pyrosulfite, or mixtures thereof as reduction agents.

Sodium persulfate and potassium pyrosulfite can be used as a redox initiator system. Likewise, sodium persulfate, potassium pyrosulfite, and iron sulfate can be used as a redox initiator system.

The additive can be present as a hydrogel.

According to the invention, the method for producing the additive comprises the following steps:
presenting the monomer in the aqueous phase,
providing the components of the redox initiator system in aqueous solutions,
adding the components of the redox initiator system in the aqueous solutions into the aqueous phase composed of the monomer, and thereby
starting the emulsion polymerization.

In this regard, a cross-linking agent can be added after presenting the monomer in the aqueous phase.

In this regard, 2-acrylamido-2-methyl propane sulfonic acid can be used as a monomer, ethylene glycol dimethacrylate or ethylene glycol dimethacrylate and N,N-methylene bisacrylamide can be used as a cross-linking agent, and sodium persulfate and potassium pyrosulfite or sodium persulfate, potassium pyrosulfite, and iron sulfate can be used as a redox initiator system.

The additive can be obtained by emulsion polymerization of 2-acrylamido-2-methyl propane sulfonic acid with ethylene glycol dimethacrylate as a cross-linking agent, and sodium persulfate, potassium pyrosulfite, and iron sulfate as a redox initiator system.

Furthermore, the additive can be obtained by emulsion polymerization of 2-acrylamido-2-methyl propane sulfonic acid with a mixture of ethylene glycol dimethacrylate and N,N-methylene bisacrylamide as a cross-linking agent, and sodium persulfate, potassium pyrosulfite, and iron sulfate as a redox initiator system.

The polymerization of the method can be started at room temperature. Room temperature means a temperature of 22° C.

According to the invention, a storage medium for a latent heat storage unit contains sodium acetate trihydrate and the additive.

According to the invention, the method for stabilization of a storage medium for a latent heat storage unit, wherein the storage medium comprises sodium acetate trihydrate and the additive, comprises the following steps:
heating of solid sodium acetate trihydrate and thereby conversion to the liquid phase,
subsequently adding the additive,
mixing the components with one another, and
cooling the product obtained to room temperature.

In this regard, the product can be cooled in controlled manner or it can be allowed to cool off in uncontrolled manner.

In the method, the solid sodium acetate trihydrate can be heated to a temperature of above 58° C., preferably to above 65° C., even more preferably to above 78° C., particularly to above 83° C., to convert it to the liquid phase.

Normally, the complete melting process only ends at approximately 77° C. At this temperature, H₂O and sodium acetate are present in separate form. With an additive according to the invention, stability can already be achieved at lower temperatures.

In the method, the additive, calculated as dry mass, can be added in an amount of up to 5 wt.-%, preferably in an amount of 0.1 to 2 wt.-%, particularly preferably in an amount of 0.5 to 1 wt.-% of the total weight of the sodium acetate trihydrate.

In this regard, each addition of H₂O or additive lowers the storable energy density of the medium, wherein the additive has a positive effect on the usable temperature. In addition, too much added water also lowers the usable temperature of the system. It is therefore preferred to make do with as little additive and added water as possible. Max. 5% additive and 8% added water can be viewed as an upper limit.

The additive, calculated as dry mass, can be added in an amount of 0.75 wt.-% or 1 wt.-%.

In the method, the additive can be present as a hydrogel. Thereby water is also added to the sodium acetate trihydrate with the hydrogel, which water is inherently contained in this hydrogel. Furthermore, additional water can be added to the sodium acetate trihydrate aside from the water inherently contained in the hydrogel.

In the method, the additional water can be added directly to the sodium acetate trihydrate, or the additional water is added to the hydrogel and then introduced into the sodium acetate trihydrate by way of the hydrogel.

In the method, the total amount of additional water added to the sodium acetate trihydrate can amount to up to 8 wt.-%, preferably up to 6 wt.-%, particularly preferably up to 5 wt.-% with reference to the weight of the sodium acetate trihydrate.

Experiments have shown that the stability of the storage medium is particularly increased if the additive, calculated as dry mass, is added in an amount of 0.75 wt.-%, and the total amount of additional water that is added to the sodium acetate trihydrate amounts to 5 wt.-%, with reference to the weight of the sodium acetate trihydrate.

Further experiments have also shown that the stability of the storage medium is particularly increased if the additive, calculated as dry mass, is added in an amount of 1 wt.-%, and the total amount of additional water that is added to the sodium acetate trihydrate amounts to 6 wt.-%, with reference to the weight of the sodium acetate trihydrate.

According to the invention, the additive is used as a stabilizer for sodium acetate trihydrate in a latent heat storage unit.

### EXEMPLARY EMBODIMENTS OF THE INVENTION

### Synthesis of the Additive:

### Example 1

In this example, 100 g 2-acrylamido-2-methyl propane sulfonic acid are presented in 120 ml water. Subsequently, 0.18 ml ethylene glycol dimethacrylate are added. After complete dissolution, 180 ml water are added, and 0.44 g sodium persulfate and 0.44 g potassium pyrosulfite are each dissolved in 20 ml water. The sodium persulfate solution and the potassium pyrosulfite solution are added to the solution of monomer and cross-linking agent, and polymerization is started. After the reaction is complete, 60 ml water are added.

### Example 2

In this example, 100 g 2-acrylamido-2-methyl propane sulfonic acid are presented in 120 ml water, and 0.23 g FeSO₄ are dissolved in this. Then 0.18 ml ethylene glycol dimethacrylate are added, and after complete dissolution, 180 ml water are added. 0.44 g sodium persulfate and 0.44 g potassium pyrosulfite are each dissolved in 20 ml water. Subsequently, the sodium persulfate solution and the potassium pyrosulfite solution are added to the solution of monomer, cross-linking agent, and iron sulfate, and polymerization is started. After the reaction is complete, 60 ml water are added.

### Use of the Additive:

### Example 3

Sodium Acetate Trihydrate with 0.75% Additive (Calculated as Dry Mass) and 5% Extra Water

The additive produced according to Example 2 is present as a hydrogel. 7.5 g of this hydrogel are filled up with 4 ml water. The resulting mixture contains 1.5 g polymer, calculated as dry mass, and 10 g water.

200 g solid sodium acetate trihydrate are heated until the material has liquefied completely. Then the additive is added and homogeneously mixed with the sodium acetate trihydrate. Subsequently, the mixture is placed in the refrigerator, cooled to 4° C., and allowed to stand in the refrigerator for 3 days. Afterward, the sample is stored at room temperature for another 3 days. The product obtained is a clear solution.

Crystallization is initiated, so that the sodium acetate trihydrate crystallized out and the latent heat is released. The released heat is conducted away and the sample is thereby cooled back down to room temperature. Subsequently, the solid sodium acetate trihydrate is heated again and thereby liquefied, and the entire cycle is repeated for a total of 6 times.

### Example 4

Sodium Acetate Trihydrate with 1% Additive (Calculated as Dry Mass) and 6% Extra Water

The additive produced according to Example 2 is present as a hydrogel. 10 g of this hydrogel are filled up with 4 ml water. The resulting mixture contains 2 g polymer, calculated as dry mass, and 12 g water.

200 g solid sodium acetate trihydrate are heated until the material is completely liquefied. Then the additive is added and homogeneously mixed with the sodium acetate trihydrate. Subsequently, the mixture is placed in the refrigerator, cooled to 4° C., and allowed to stand in the refrigerator for 3 days. Afterward, the sample is stored at room temperature for another 3 days. The product obtained is a clear solution.

Crystallization is initiated, so that the sodium acetate trihydrate crystallizes out and the latent heat is released. The released heat is conducted away and the sample is thereby cooled back to room temperature. Subsequently, the solid sodium acetate trihydrate is heated again and thereby liquefied, and the total cycle is repeated for a total of 6 times.

1. An additive for stabilization of sodium acetate trihydrate, which can be obtained by means of emulsion polymerization of at least one hydrophilic monomer selected from acrylic acid and acrylic acid derivatives with an acid group, and a redox initiator system.

2. The additive according to claim 1, wherein the monomer comprises an acrylic acid derivative of sulfonic acid or of phosphonic acid or a salt of sulfonic acid or phosphonic acid.

3. The additive according to claim 1, wherein the monomer is selected from the group comprising 2-acrylamido-2-methyl propane sulfonic acid, 2-methacrylamido-2-methyl propane sulfonic acid, 2-acrylamido-2-methyl-1-propane phosphonic acid, 2-methacrylamido-2-methyl-1-propane phosphonic acid, 2-(methacryloyloxy)ethyl-phosphonic acid, a salt thereof, or a mixture thereof.

4. The additive according to claim 1, wherein the monomer is 2-acrylamido-2-methyl propane sulfonic acid.

5. The additive according to claim 1, wherein the additive was obtained using a cross-linking agent.

6. The additive according to claim 5, wherein the cross-linking agent is selected from the group comprising ethylene glycol dimethyl acrylate, N,N-methylene bisacrylamide, pentaerythritol tetra-acrylate, trimethylolpropane trimethacrylate, trimethylolpropane triacrylate, and mixtures thereof.

7. The additive according to claim 1, wherein redox initiator system comprises a persulfate, a hydroperoxide or an Na or K hypochloride as an oxidation agent, and ascorbic acid, formaldehyde sulfoxylate, tetramethylene diamine, sodium hydroxymethylsulfinate, potassium pyrosulfite, sodium hydrogen sulfite, sodium pyrosulfite, or mixtures thereof as reduction agents.

8. The additive according to claim 1, wherein sodium persulfate and potassium pyrosulfite are used as a redox initiator system, or sodium persulfate, potassium pyrosulfite, and iron sulfate are used as a redox initiator system.

9. The additive according to claim 1, wherein the additive is present as a hydrogel.

10. A method for producing the additive according to claim 1,
wherein the method comprises the following steps:
presenting the monomer in the aqueous phase,
providing the components of the redox initiator system in aqueous solutions,
adding the components of the redox initiator system in the aqueous solutions into the aqueous phase composed of the monomer, and thereby
starting the emulsion polymerization.

11. The method according to claim 10, wherein a cross-linking agent is added to the aqueous phase after presenting the monomer in the aqueous phase.

12. The method according to claim 10, wherein 2-acrylamido-2-methyl propane sulfonic acid is used as a monomer, ethylene glycol dimethacrylate or ethylene glycol dimethacrylate and N,N-methylene bisacrylamide is used as a cross-linking agent, and sodium persulfate and potassium pyrosulfite are used as a redox initiator system, or sodium persulfate, potassium pyrosulfite, and iron sulfate are used as a redox initiator system.

13. The method according to claim 10, wherein the polymerization is started at room temperature.

14. A storage medium for a latent heat storage unit, wherein the storage medium comprises sodium acetate trihydrate and the additive according to claim 1.

15. A method for stabilization of a storage medium for a latent heat storage unit, wherein the storage medium comprises sodium acetate trihydrate and the additive according to claim 1, and comprises the following steps:
heating of solid sodium acetate trihydrate and thereby conversion to the liquid phase,
subsequently adding the additive according to claim 1,
mixing the components with one another, and
allowing the product obtained to cool off to room temperature.

16. The method according to claim 15, wherein the solid sodium acetate trihydrate is heated to a temperature of above 58° C., preferably to above 65° C., even more preferably to above 78° C., particularly preferably to above 83° C. for conversion to the liquid phase.

17. The method according to claim 15, wherein the additive, calculated as dry mass, is added in an amount of up to 5 wt.-%, preferably in an amount of 0.1 to 2 wt.-%, particularly preferably in an amount of 0.5 to 1 wt.-% of the total weight of the sodium acetate trihydrate.

18. The method according to claim 15, wherein the additive is present as a hydrogel, and wherein additional water is added to the sodium acetate trihydrate aside from the water inherently contained in the hydrogel.

19. The method according to claim 18, wherein the additional water is added directly to the sodium acetate trihydrate, or the additional water is added to the hydrogel and then introduced into the sodium acetate trihydrate by way of the hydrogel.

20. The method according to claim 18, wherein the total amount of additional water added to the sodium acetate trihydrate amounts to up to 8 wt.-%, preferably up to 6 wt.-%, particularly preferably up to 5 wt.-% with reference to the weight of the sodium acetate trihydrate.

21. Use of the additive according to claim 1 as a stabilizer for sodium acetate trihydrate in a latent heat storage unit.
